(19) United States
(12) Patent Application Publication
(10) Pub. No.: US 2026/0335362 A1
(43) Pub. Date: Oct. 29, 2026

(54) ELEMENTS AND SYSTEM FOR WOUND TREATMENT

(71) Applicant: REDDRESS LTD., Pardes Hana (IL)

(72) Inventors: Alon KUSHNIR, Givat Ada (IL); Ohad ILAN, Givat Ada (IL)

(21) Appl. No.: 19/121,288

(22) PCT Filed: Oct. 16, 2023
(86) PCT No.: PCT/IL2023/051081

§ 371 Date: Apr. 15, 2025

(30) Foreign Application Priority Data
Oct. 18, 2022 (IL) 297401

(51) Int. Cl. A61K 35/14; A61M 5/31; A61M 35/00

(52) U.S. Cl. CPC A61K 35/14; A61M 5/3147; A61M 35/00

(57) ABSTRACT

Coagulating blood is produced that can be used for treatment of wounds, for example ulcers, abnormal sinuses fistulae. The coagulating blood may then be applied onto or into the wound to thereby treat the wound. Whole blood is transferred inti a syringe containing a coagulating agent through the use of a connector that facilitates such transfer. The coagulating blood can then be transferred into the wound.

### TECHNICAL FIELD

The present disclosure relates to a field of medical devices. More particularly, the present disclosure relates to systems, methods and kits for use in wound treatment.

### BACKGROUND

The information in this section merely provides background information related to the present disclosure and may not constitute prior art(s) for the present disclosure.

References considered to be relevant as background to the present disclosure are listed below:
U.S. Pat. No. 9,180,142
U.S. Pat. No. 10,111,979
WO 2019/058375
WO 2019/150355
WO 2021/205457
WO 2022/168099

Acknowledgement of the above references herein is not to be inferred as meaning that these are in any way relevant to the patentability of the device/system/method disclosed in the present disclosure.

US patent publications 9,180,142 and 10,111,979 disclose a wound treatment procedure by which blood is coagulated and the so-formed blood coagulant is applied onto a wound with a dressing material. Further, PCT publications WO 2019/058373, WO 2019/058375 and WO 2019/150355 disclose methods, devices, and systems for dressing wounds with a blood coagulant-comprising dressing. The blood clot-comprising dressing is, typically, prepared from blood taken from a patient. As the blood sample to be withdrawn is of a relatively small amount (as compared to that of a blood donation to a blood bank), e.g., 10-20 ml, the blood sample is typically drawn into a vacuum-holding test tube that is sealed by an elastic, typically rubber, stopper, of the kind used in blood tests, and then has to be withdrawn from the test tube. As the test tube is sealed, the blood withdrawal, typically with a syringe has to be against the vacuum inside the tube and this may be challenging.

PCT publication WO 2021/205457 discloses a device that allows extraction of the blood from a test tube by pumping a gas, e.g., air, into the test tube through one conduit, whereupon blood is extracted out of another conduit into a blood coagulation molding device. Use of coagulated blood mass in the healing process of an abnormal hollowed space, such as an annal fistula. Method and a kit for treating an abnormal hollowed space of a subject, such as a fistula, pilonidal sinus or a stoma are disclosed in PCT publication WO 2022/168099.

Withdrawing blood from a test tube by a syringe is challenging as there is a need to overcome the vacuum that develops within the test tube.

### SUMMARY

In accordance with this disclosure coagulating blood is produced that can be used for treatment of wounds, for example ulcers, abnormal sinuses fistulae. The coagulating blood may then be applied onto or into the wound to thereby treat the wound. Provided by this disclosure is a system for preparing a coagulating blood mass, a system for use in wound treatment and corresponding methods for preparing a coagulating blood mass and for wound treatment. Also provided by this disclosure is a kit of parts and a syringe that may be used in the methods of this disclosure.

The term “coagulating blood” is used herein to denote whole blood that is still in a flowable state and is in the process of coagulation. Coagulating blood is obtained by mixing whole blood mixed a coagulating agent. A coagulating blood is converted within several minutes into a coagulated blood mass.

The term “coagulating agent” is used herein to denote (i) one or more agents that induce blood coagulation, (ii) or one or more anti-anticoagulants, namely agents that counteract the action of an anticoagulant that may be mixed with the withdrawn blood, or (iii) a combination of one or more agents that induce blood coagulation and one or more anti-anticoagulants.

The term “coagulated blood mass” is used herein to note the mass of coagulated blood formed form the coagulating blood.

The term “subject” is used herein to denote a human or non-human animal to be treated in accordance with this disclosure.

In accordance with an aspect of the present disclosure, a system for preparing a coagulating blood mass is disclosed. This system comprises a syringe accommodating a blood coagulating agent and a connector device. The connector device comprises an adapter body having (i) a test tube port for coupling with a test tube, (ii) a syringe port for coupling with a syringe, and (iii) a gas inlet port. The connector device further comprises at least one first fluid conduit extending through the adapter body between the test tube port and the syringe port. The at least one first fluid conduit comprises a first end projection at the test tube port configured for piercing through a stopper of the test tube. The connector device furthermore comprises at least one second fluid conduit extending through the adapter body between the test tube port and the gas inlet port. The at least one second fluid conduit comprises a second end projection at the test tube port configured for piercing through the stopper of the test tube. When a test tube is coupled to the test tube port and a syringe is coupled to the syringe port, the at least one first fluid conduit enables fluid communication between the test tube and the syringe, and the at least one second fluid conduit enables fluid communication between the test tube and the gas inlet port.

In accordance with a further aspect of the present disclosure, a wound treatment system is disclosed. The wound treatment system comprises a syringe accommodating a coagulating agent. The wound treatment system further comprises the connector device noted above, for coupling with the test tube and the syringe to permit drawing the blood mass from the test tube into the syringe to thereby mix the drawn blood mass with the coagulating agent to form a coagulating blood mass. The wound treatment system further comprises an elongated application tube or a needle configured for coupling with the syringe and for transferring the coagulating blood mass to a site of need in a body of a subject.

In one non-limiting embodiment the present disclosure, the above systems comprise a test tube intended to contain a volume of blood mass. The test tube may be a vacuum-holding container, know also in the art as “vacutainer”.

In one non-limiting embodiment of the present disclosure, the connector device comprises a test tube adapter at the test tube port configured to facilitate coupling of the test tube port with the test tube. The connector device further comprises a syringe adapter at the syringe port configured to facilitate coupling of the syringe port with the syringe.

In another non-limiting embodiment of the present disclosure, the test tube port and the syringe port are oppositely disposed. However, this is but an example of the many relative disposition options of the test tube port and the syringe port.

In another non-limiting embodiment of the present disclosure, the test tube adapter is aligned co-axially with the syringe adapter.

In another non-limiting embodiment of the present disclosure, the syringe port comprises a Luer-type fitment for coupling with a counterpart Luer-type fitment of the syringe.

In another non-limiting embodiment of the present disclosure, the test tube port is fitted with a test tube adapter in the form of a hollow cylinder extending between the test tube port and an open end. Further, the first and second end projections of the at least one first fluid conduit and the at least one second fluid conduit, respectively, project into an interior of the hollow cylinder. Furthermore, the test tube adapter is being configured to receive a stopper-sealed end of the test tube in a manner permitting the first and second end projections to pierce the stopper of the test tube.

In another non-limiting embodiment of the present disclosure, the first and the second end projections comprise a tapered or sharpened tip for piercing the stopper of the test tube.

In another non-limiting embodiment of the present disclosure, the gas inlet port defines a channel in fluid communication with the at least one second fluid conduit.

In another non-limiting embodiment of the present disclosure, the gas inlet port comprises a filter for filtering gas flowing into the gas inlet port.

In another non-limiting embodiment of the present disclosure, the at least one first fluid conduit and the at least one second fluid conduit are arranged parallel to each other.

In another non-limiting embodiment of the present disclosure, one or both of the at least one first fluid conduit or the at least one second fluid conduit are formed of metal tube.

In another non-limiting embodiment of the present disclosure, the coagulating agent contained in the syringe is a dry particulate matter. The coagulating agent may also be one or more of (i) first agents that induce blood coagulation, (ii) one or more second anti-anticoagulants that counteracts action of anti-coagulants. For example, the coagulating agent may be one or more of the groups consisting of Kaolin, Calcium salts, Magnesium salts, negatively charged phospholipid and protamine sulfate.

In another non-limiting embodiment, the test tube holds a vacuum.

The coagulating blood mass that is produced by the use of the above system, may then be used for treating a wound, as will described below.

In accordance with another aspect of the present disclosure, a method of preparing a coagulating a blood mass is disclosed. The method comprises coupling a syringe containing a coagulating agent to the syringe port of the connector device as disclosed above. The method further comprises coupling a test tube containing a volume of the blood mass to the test tube port of the connector device, such that the first and second end projections of the at least one first fluid conduit and the at least one second fluid conduit, respectively, pierce a stopper of the test tube. The method further comprises drawing the blood mass from the test tube into the syringe, through the at least one first fluid conduit, for mixing the blood mass with the coagulating agent to form a coagulating blood mass.

In accordance with a yet further aspect of the present disclosure, a method of treating a wound is disclosed. The method comprises coupling a syringe containing a coagulating agent to the syringe port of the connector device as disclosed above. The method further comprises coupling a test tube containing a volume of the blood mass to the test tube port of the connector device, such that the first and second end projections of the at least one first fluid conduit and the at least one second fluid conduit, respectively, pierce a stopper of the test tube. The method furthermore comprises drawing the blood mass from the test tube into the syringe, through the at least one first fluid conduit, for mixing the blood mass with the coagulating agent to form a coagulating blood mass. Further, the method comprises transferring the coagulating blood mass to a site of need in a body of a subject.

In a non-limiting embodiment, in the above methods use is made by the above-described systems.

In a non-limiting embodiment of the present disclosure, the methods comprise, prior to coupling the test tube with the connector device, withdrawing the blood mass from a subject and transferring the blood mass to the test tube. The test tube may, for example, be a vacuum-containing tube into which blood is withdrawn directly from the subject.

In a non-limiting embodiment of the method for treating a wound of the present disclosure, transferring the coagulating blood mass to the site of need in the body of the subject comprises transferring the coagulating blood mass to said site through an elongated application tube or a needle configured for coupling with the syringe.

In another non-limiting embodiment of the present disclosure, the methods comprise, prior to coupling the test tube with the connector device, withdrawing the blood mass from the subject and transferring the blood mass to the test tube.

In accordance with another aspect of the present disclosure, a kit of parts is disclosed. The kit of parts comprises a syringe containing a coagulating agent, as described above, the connector device as disclosed above, and an elongated application tube or a needle configured to be coupled to the syringe for transferring a coagulating blood mass to a site of need in a body of a subject.

The kit in accordance with non-limiting embodiments of this disclosure comprises elements of the non-limiting disclosure described above.

It is to be understood that the aspects and embodiments of the disclosure described above may be used in any combination with each other. Several of the aspects and embodiments may be combined together to form a further embodiment of the disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### EMBODIMENTS

Some non-limiting embodiments of this disclosure are listed, each one beginning with a number indicating its serial the number in this list. These embodiments do not limit the generality of the disclosure herein.

1. A system for preparing a coagulating blood volume, comprising:
a syringe accommodating a blood coagulating agent; and
a connector device that comprises
an adapter body having (i) a test tube port for coupling with a test tube containing a whole blood mass, (ii) a syringe port for coupling with the syringe, and (iii) a gas inlet port;
at least one first fluid conduit extending through the adapter body between the test tube port and the syringe port, the at least one first fluid conduit comprising a first end projection at the test tube port configured for piercing through a stopper of the test tube; and
at least one second fluid conduit extending through the adapter body between the test tube port and the gas inlet port, the at least one second fluid conduit comprising a second end projection at the test tube port configured for piercing through the stopper of the test tube;
when a test tube is coupled to the test tube port and a syringe is coupled to the syringe port, the at least one first fluid conduit enables fluid communication between the test tube and the syringe, and the at least one second fluid conduit enables fluid communication between the test tube and the gas inlet port.
   2. The system of embodiment 1, comprising a test tube for containing a volume of whole blood.
   3. The system of embodiment 1 or 2, comprising:
a test tube adapter at the test tube port configured to facilitate coupling of the test tube port with the test tube; and
a syringe adapter at the syringe port configured to facilitate coupling of the syringe port with the syringe.
   4. The system of embodiments 1-3, wherein the test tube port and the syringe port are oppositely disposed.
   5. The system of embodiments 4, wherein the test tube adapter is aligned co-axially with the syringe adapter.
   6. The system of any one of embodiments 1 to 5, wherein the syringe port comprises a Luer-type fitment for coupling with a counterpart Luer-type fitment of the syringe.
   7. The system as claimed in any one of embodiments 1 to 6, wherein
the test tube port is fitted with a test tube adapter in the form of a hollow cylinder extending between the test tube port and an open end,
the first and second end projections of the at least one first fluid conduit and the at least one second fluid conduit, respectively, project into an interior of the hollow cylinder, and
the test tube adapter is being configured to receive a stopper-sealed end of the test tube in a manner permitting the first and second end projections to pierce the stopper of the test tube.
   8. The system as claimed in any one of embodiments 1 to 7, wherein the first and the second end projections comprise a tapered or sharpened tip for piercing the stopper of the test tube.
   9. The system of any one of embodiments 1 to 8, wherein
the gas inlet port defines a channel in fluid communication with the at least one second fluid conduit.
   10. The system of one of embodiments 1 to 9, wherein the gas inlet port comprises a filter for filtering gas flowing into the gas inlet port.
   11. The system of one of embodiments 1 to 10, wherein the at least one first fluid conduit and the at least one second fluid conduit are arranged parallel to each other.
   12. The system of one of embodiments 1 to 11, wherein one or both of the at least one first fluid conduit or the at least one second fluid conduit are formed of metal tube.
   13. The system of any one of embodiments 1 to 12, wherein the coagulating agent contained in the syringe is a dry particulate matter.
   14. The system of any one of embodiments 1 to 13, wherein the coagulating agent is one or more of the groups consisting of Kaolin, Calcium salts, Magnesium salts, negatively charged phospholipid and protamine sulfate.
   15. The system of any one of embodiments 1 to 14, wherein the coagulating agent is one or more first agents that induce blood coagulation and/or one or more second anti-anticoagulants that counteracts action of anti-coagulants.
   16. The system of any one of embodiments 1 to 15, wherein the test tube holds a vacuum.
   17. A method for preparing a coagulating a blood mass, comprising:
coupling a syringe containing a coagulating agent to a syringe port of a connector device that comprises
an adapter body having (i) a test tube port for coupling with a test tube containing a whole blood mass, (ii) a syringe port for coupling with the syringe, and (iii) a gas inlet port,
at least one first fluid conduit extending through the adapter body between the test tube port and the syringe port, the at least one first fluid conduit comprising a first end projection at the test tube port configured for piercing through a stopper of the test tube, and comprises
at least one second fluid conduit extending through the adapter body between the test tube port and the gas inlet port, the at least one second fluid conduit comprising a second end projection at the test tube port configured for piercing through the stopper of the test tube; coupling a test tube containing a volume of blood to the test tube port of the connector device, such that the first and second end projections of the at least one first fluid conduit and the at least one second fluid conduit, respectively, pierce a stopper of the test tube; and comprising
drawing the blood mass from the test tube into the syringe, through the at least one first fluid conduit, for mixing the blood mass with the coagulating agent to form a coagulating blood mass.
   18. The method of embodiment 13, comprising, prior to coupling the test tube with the connector device,
withdrawing blood from a subject and transferring the blood to the test tube.
   19. The method of embodiment 17 or 18, making use of the system as claimed in any one of embodiments 1 to 16.
   20. A wound treatment system, comprising:
a syringe accommodating a coagulating agent;
a connector device that comprises
an adapter body having (i) a test tube port for coupling with a test tube containing a whole blood mass, (ii) a syringe port for coupling with the syringe, and (iii) a gas inlet port,
at least one first fluid conduit extending through the adapter body between the test tube port and the syringe port, the at least one first fluid conduit comprising a first end projection at the test tube port configured for piercing through a stopper of the test tube, and comprises
at least one second fluid conduit extending through the adapter body between the test tube port and the gas inlet port, the at least one second fluid conduit comprising a second end projection at the test tube port configured for piercing through the stopper of the test tube;
an elongated application tube or a needle configured for coupling with the syringe and for transferring the coagulating blood mass to a site of need in a body of a subject.
   21. The system of embodiment 20 a test tube to contain a volume of the blood mass.
   22. The system of embodiment 20 or 21, comprising:
one or more blood mass collection elements for withdrawing the blood mass from the subject and transferring the blood mass to the test tube.
   23. The system of any one of embodiments 20 to 22, wherein the coagulating agent contained in the syringe is a dry particulate matter.
   24. The system of any one of embodiments 20 to 23, wherein the coagulating agent is one or more of the groups consisting of Kaolin, Calcium salts, Magnesium salts, negatively charged phospholipid and protamine sulfate.
   25. The system of any one of embodiments 20 to 24, wherein the coagulating agent is one or more first agents that induce blood coagulation and/or one or more second anti-anticoagulants that counteracts action of anti-coagulants.
   26. The system of any one of embodiments 20 to 25, wherein the test tube holds a vacuum.
   27. A method of treating a wound, comprising:
coupling a syringe containing a coagulating agent to the syringe port of the connector device that comprises
an adapter body having (i) a test tube port for coupling with a test tube containing a whole blood mass, (ii) a syringe port for coupling with the syringe, and (iii) a gas inlet port,
at least one first fluid conduit extending through the adapter body between the test tube port and the syringe port, the at least one first fluid conduit comprising a first end projection at the test tube port configured for piercing through a stopper of the test tube, and comprises
at least one second fluid conduit extending through the adapter body between the test tube port and the gas inlet port, the at least one second fluid conduit comprising a second end projection at the test tube port configured for piercing through the stopper of the test tube;
coupling a test tube containing a volume of blood to the test tube port of the connector device, such that the first and second end projections of the at least one first fluid conduit and the at least one second fluid conduit, respectively, pierce a stopper of the test tube;
drawing the blood mass from the test tube into the syringe, through the at least one first fluid conduit, for mixing the blood mass with the coagulating agent to form a coagulating blood mass; and comprising
transferring the coagulating blood mass to a site of need in a body of a subject.
   28. The method of embodiment 27, wherein transferring the coagulating blood mass in the body of the subject comprises transferring the coagulating blood mass through an elongated application tube or a needle configured for coupling with the syringe.
   29. The method of embodiment 27 or 28, comprising, prior to coupling the test tube with the connector device,
withdrawing the blood mass from the subject and transferring the blood mass to the test tube.
   30. A kit of parts, comprising:
a syringe containing a coagulating agent;
a connector device that comprises
an adapter body having (i) a test tube port for coupling with a test tube containing a whole blood mass, (ii) a syringe port for coupling with the syringe, and (iii) a gas inlet port,
at least one first fluid conduit extending through the adapter body between the test tube port and the syringe port, the at least one first fluid conduit comprising a first end projection at the test tube port configured for piercing through a stopper of the test tube, and comprises
at least one second fluid conduit extending through the adapter body between the test tube port and the gas inlet port, the at least one second fluid conduit comprising a second end projection at the test tube port configured for piercing through the stopper of the test tube; and comprising
an elongated application tube or a needle configured to be coupled to the syringe for transferring a coagulating blood mass to a site of need in a body of a subject.
   31. The kit of embodiment 30, comprising:
a test tube for holding a volume of blood mass withdrawn from the subject.
   32. The kit of embodiments 30 or 31, comprising:
one or more blood collection elements for withdrawing the blood mass from the subject and transferring the blood mass to the test tube.
   33. The kit of any one of embodiments 30 to 32, wherein the coagulating agent contained in the syringe is a dry particulate matter.
   34. The kit of any one of embodiments 30 to 33, wherein the coagulating agent is one or more of groups consisting of Kaolin, Calcium salts, Magnesium salts, negatively charged phospholipid and protamine sulfate.
   35. The kit of any one of embodiments 30 to 34, wherein the coagulating agent is one or more first agents that induce blood coagulation and/or one or more second anti-anticoagulants that counteracts action of anti-coagulants.
   36. A syringe, comprising:
a hollow syringe barrel with a first end configured to have a needle fixed thereto;
a plunger provided at a second end of the hollow syringe barrel, the plunger configured to be in sliding engagement with walls of the hollow syringe barrel; and
a coagulating agent contained in the hollow syringe barrel.
   37. The syringe of embodiment 36, wherein the coagulating agent contained in the syringe is a dry particulate matter.
   38. The syringe of embodiment 36 or 37, wherein the coagulating agent is one or more of groups consisting of Kaolin, Calcium salts, Magnesium salts, negatively charged phospholipid and protamine sulfate.
   39. The syringe of any one of embodiments 36 to 38, wherein the coagulating agent is one or more first agents that induce blood coagulation and/or one or more second anti-anticoagulants that counteracts action of anti-coagulants.
   40. The system of any one of embodiments 1 to 16 for use in the method of any one of embodiments 17 to 19.
   41. The system of any one of embodiments 17 to 26 for use in the method of any one of embodiments 27 to 29.
   42. The kit of embodiments 30 to 35 for use in the method of any one of embodiments 27 to 29.
   43. The syringe of embodiments 36 to 39, for use (i) in the system of any one of embodiments 1 to 16 and 17 to 26, (ii) in the method of any one of embodiments 17 to 19 or 27 to 29, or for the use is (iii) the kit of any one of embodiments 30 to 35.

### BRIEF DESCRIPTION OF FIGURES

Embodiments of this disclosure are now described, by way of example only, with reference to the accompanying schematic drawings, wherein like reference numerals represent like elements and in which:

FIG. 1 is a front view of a connector device, useful in some embodiments of the present disclosure;

FIG. 2 is a front view of the connector device of FIG. 1, without a cylindrical housing of an adapter body of the connector device, in accordance with another embodiment of the present disclosure;

FIG. 3 is a longitudinal cross-sectional view of the connector device of FIG. 2, along with an enlarged view of a gas inlet port of the connector device, in accordance with an embodiment of the present disclosure;

FIG. 4 is a front view of a syringe, in accordance with an embodiment of the present disclosure;

FIG. 5 is a front view of a system for preparing a coagulating blood mass, the system comprising the connector device of FIG. 1, in which the connector device is coupled with a test tube, and the syringe of FIG. 4, in accordance with an embodiment of the present disclosure;

FIG. 6 is another front view of the system of FIG. 5 after some blood has been withdrawn from the test tube into the syringe, in accordance with an embodiment of the present disclosure;

FIG. 7 is a cross-sectional view of the connector device of FIG. 2 coupled with the test tube, in accordance with an embodiment of the present disclosure; and

FIG. 8 is a front view of the syringe of FIG. 4, to which an elongated application tube for transferring contents contained in the syringe, is coupled, in accordance with an embodiment of the present disclosure.

Skilled artisans will appreciate that elements in the drawings are illustrated for simplicity and have not necessarily been drawn to scale. For example, the dimensions of some of the elements in the drawings may be exaggerated relative to other elements to help to improve understanding of embodiments of the present disclosure.

### DETAILED DESCRIPTION OF EXEMPLARY EMBODIMENTS

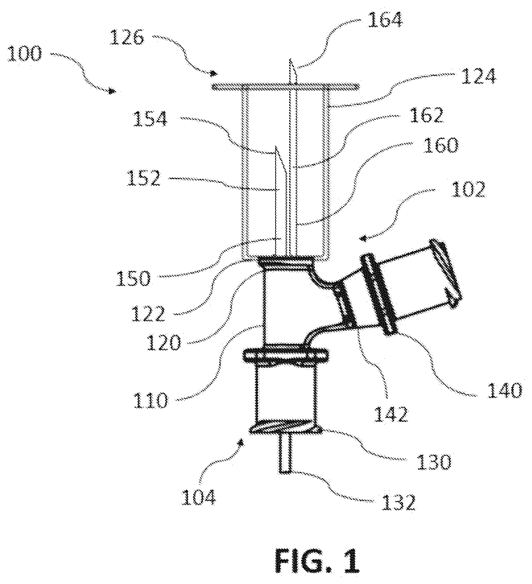

While the disclosure is susceptible to various modifications and alternative forms, specific embodiments thereof have been shown by way of example in FIGS. 1-8 and will be described in detail below. It should be understood, however that it is not intended to limit the present disclosure to the particular forms disclosed, but on the contrary, the present disclosure is to cover all modifications, equivalents, and alternatives falling within the general scope of the present disclosure as defined by the appended claims.

It is to be noted that a person skilled in the art can be motivated from the present disclosure and modify the various constructions of the system for coagulating blood, a wound treatment system, a kit and a syringe for preparing a coagulating blood mass, all being within the general scope of the teaching of the present disclosure. Accordingly, the drawings are showing only those specific details that are pertinent to understanding the embodiments of the present disclosure so as not to obscure the disclosure with details that will be readily apparent to those of ordinary skill in the art having benefit of the description herein.

In the present disclosure, the term “exemplary” or the like, is used herein to mean “serving as an example, instance, or illustration.” Any embodiment or implementation of the present subject matter described herein as “exemplary” is not necessarily to be construed as preferred or advantageous over other embodiments.

In the present disclosure, the terms “comprises”, “comprising”, or any other variations thereof, are intended to cover a non-exclusive inclusions, such that a device that comprises a list of components does not include only those components but may include other components not expressly listed or inherent to such setup or device. In other words, one or more elements in a system or apparatus proceeded by “comprises . . . a” does not, without more constraints, preclude the existence of other elements or additional elements in the system or apparatus.

In the present disclosure, the terms like “at least one” and “one or more” may be used interchangeably or in combination throughout the description.

Reference will now be made to the exemplary embodiments of the disclosure, as schematically illustrated in the accompanying drawings. Wherever possible, same numerals will be used to refer to the same or like parts. Embodiments of the disclosure are described in the following paragraphs with reference to FIGS. 1 to 8. In FIGS. 1 to 8, the same element or elements which have same functions are indicated by the same reference signs.

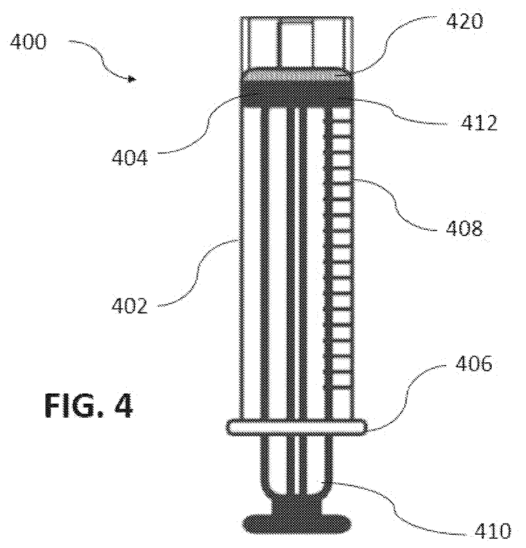

Referring to FIG. 1, a connector device 100 is shown that is part of a system that also includes the syringe, shown in FIG. 4. In an embodiment, the connector device 100 is adapted to couple with a test tube containing a blood mass, at a first end 102 of the connector device 100, in a manner to be explained below. The connector device 100 is further adapted to couple with a syringe containing a coagulating agent, such as syringe 400 of FIG. 4, at a second end 104 of the connector device 100. In said embodiment, when the connector device 100 is coupled with the test tube and the syringe, the connector device 100 facilitates a fluid communication between the test tube and the syringe, such that the blood mass contained in the test tube is drawn into the syringe, by a movement of a plunger of the syringe, to mix at least a portion of the blood mass with the coagulating agent contained in the syringe, and form a coagulating blood mass.

The coagulating blood mass formed in the syringe may be applied or injected onto a wound for treating the wound by forming a blood coagulant in or on the wound. Without derogating from the general teaching of the present disclosure, a subject may be a patient suffering from the illness or wound, and may be a human or a non-human animal. Further, the wound may be a wound formed on the surface of a skin or another exposed surface, may be a penetrating wound, such as an abnormal cavity that is formed in human or non-human animal due to or in a result of a certain medical condition. For example, the wound may be a fistula, e.g., an anal fistula, a stoma opening, or a pilonidal sinus. Also, the wound may be naturally occurring as a result of a certain medical condition, may be a consequence of an injury, may be a by-product of a surgical procedure, etc.

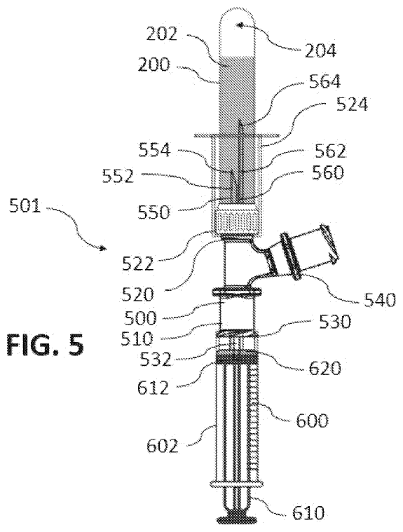

In accordance with the present disclosure, the blood mass is whole blood, typically, an autologous blood, withdrawn from the subject. The blood mass is collected in a blood-collection test tube that typically holds a vacuum, maintained by a stopper of the test tube. An exemplary test tube 200 containing the blood mass 202 is shown in FIG. 5.

Further, without derogating from the general teaching of the present disclosure, FIG. 4 illustrates an exemplary syringe 400. The syringe 400 is adapted to be coupled with the connector device 100, the coupling being, for example, as illustrated, through a Luer fitting—the syringe having an exemplary illustrated female-type Luer adapter for coupling to an illustrated male-type Luer adapter of a syringe port of the connector device 100. As can be appreciated, in other embodiments the syringe may have a male-type Luer adapter and the syringe port a female one; or the coupling may be by a different type of fitment. The syringe 400, shown in FIG. 4, comprises a hollow syringe barrel 402 with a front end 404 and an opposite rear end 406. A plunger 410, with a piston 412 is disposed within the syringe barrel 402, the plunger 410 typically made of or comprises an elastomeric material to secure a fluid-tight seal with walls 408 of the syringe barrel 410. Further, the front end 404 of the syringe 400 is also configured to be coupled with a needle or an application tube, by a similar type of fitment as noted above. Said needle may be any suitable needle used in medical procedures for applying or injecting or transferring or administering contents contained in the syringe barrel 402 to the subject. In some embodiments, the front end 404 of the syringe barrel 402 is configured to be coupled or fixed with an elongated application tube, e.g., a plastic tube, suitable for applying or injecting or transferring or administering the contents contained in the syringe barrel 402 to the subject or the patient.

Referring again to FIG. 4, the syringe 400 further comprises a coagulating agent 420. The coagulating agent 420 is contained in the syringe barrel 402 between the piston 412 and the front end 404 of the syringe barrel 402, as shown in FIG. 4. The coagulating agent 420 may be a dry particulate matter, for example, a finely ground or milled solid, a powder form, a lyophilizate, etc. The coagulating agent 420 may be one or more first agents that induce blood coagulation and/or one or more second anti-anticoagulants that counteracts action of anti-coagulants. Exemplary coagulating agents 420 include Kaolin, Calcium salts, Magnesium salts, negatively charged phospholipid and protamine sulfate.

Referring again to FIG. 1, the connector device 100 is shown. The connector device 100 comprises an adapter body 110, a test tube port 120, a syringe port 130 and a gas inlet port 140. Within the general scope of teachings of the present disclosure, the test tube port 120 is adapted to be coupled with a stopper-sealed test tube, for example, the test tube 200, seen in

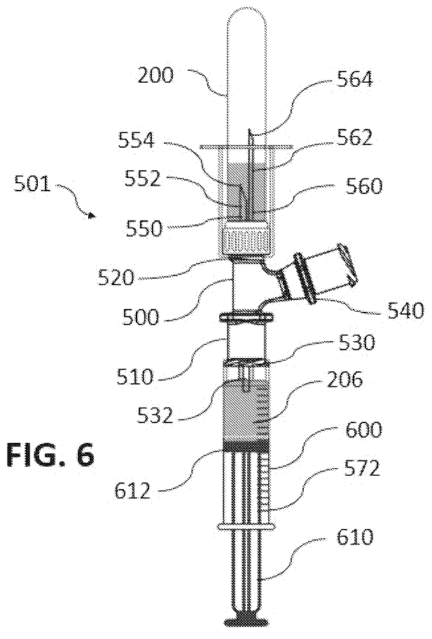

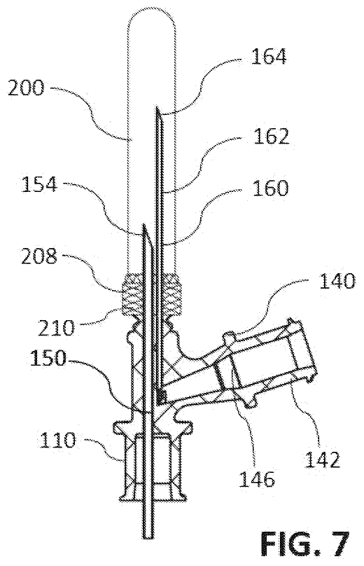

FIGS. 5 and 6. In an embodiment, the adapter body 110 comprises a test tube adapter 122 at the test tube port 120 that is configured to facilitate coupling of the test tube port 120 with the test tube 200. The test tube 200, as can be seen in FIGS. 5 and 6, is a stopper-sealed test tube that includes or has a closure 208, as seen in FIG. 7, that includes an elastomeric stopper 210, seen in FIG. 7, sealing the test tube's opening, typically, but not exclusively, made from rubber. The test tube 200 that may contain a blood mass (for example, the blood mass 202) under vacuum 204 held by the stopper 210. In an embodiment, the test tube adapter 122 is formed in the general shape of a hollow cylinder 124 having an open end 126. In this embodiment, the inner diameter of cylinder 124 corresponds to the outer diameter of the closure 208, which ensure tight fitting when the test tube 200 is coupled with the connector device 100, and hence supports the test tube 200 and holds it in a desired position.

The syringe port 130 is adapted to be coupled with a syringe, for example, the syringe 400, as shown in FIG. 4. In an embodiment, the adapter body 110 comprises a syringe adapter 132 at the syringe port 130. The syringe adapter 132 is configured to facilitate coupling of the syringe port 130 with the syringe 400, for example it may comprise a Luer-type fitment for coupling with a counterpart Luer-type fitment of the syringe 400.

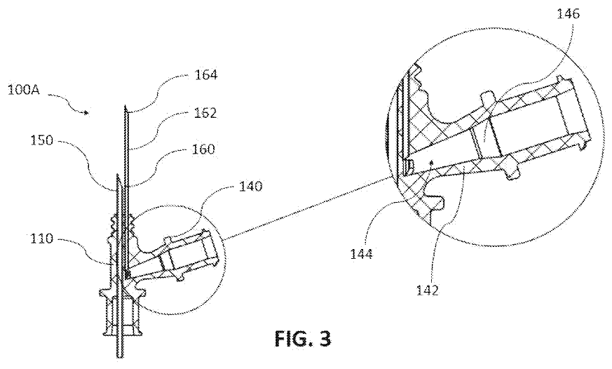

The gas inlet port 140 opens to the atmosphere. If desired, it may also be coupled with an external source of gas (not shown). The gas inlet port 140, that can best be seen in FIG. 3, defines a channel 144 within a body 142. The channel 144 is fluidically linked at its inner end to a second fluid conduit 160 of the connector device 100. Further, in some embodiments, the gas inlet port 140 comprises a filter 146 for filtering inflowing gas. In the illustrated embodiment, the gas inlet port 140 includes a Luer-type fitment while there may be other type of fitments in other embodiments or where port 140 intends to be open to the external atmosphere, it may include no fitment at all.

The connector device 100 further comprises at least one first fluid conduit 150 that extends through the adapter body 110 between the test tube port 120 and the syringe port 130. In an exemplary embodiment shown in FIGS. 1 to 3, the at least one first fluid conduit 150 is embodied as one first fluid conduit 150 that comprises a first end projection 152 at the test tube port 120 of the adapter body 110, configured to pierce through a pierceable seal of an entity, when the entity is being coupled to the connector device 100. For example, the first end projection 152 of the at least one first fluid conduit 150 is configured to pierce through the elastomeric stopper 210 of the test tube 200, when the test tube 200 is being coupled to the connector device 100. In an embodiment, the first end projection 152 comprises a tapered or sharpened tip 154 for piercing the elastomeric stopper 210 of the test tube 200. In a further embodiment, the first end projection 152 comprises a bevelled end, for example, shaped like a needle, for piercing the elastomeric stopper 210 of the test tube 200.

The connector device 100 further comprises at least one second fluid conduit 160 that extends through the adapter body 110 between the test tube port 120 and the gas inlet port 140. In the exemplary embodiment shown in FIGS. 1 to 3, the at least one second fluid conduit 160 is embodied as one second fluid conduit 160. In accordance with the present disclosure, the at least one second fluid conduit 160 comprises a second end projection 162 formed at the test tube port 120 of the adapter body 110. The second end projection 162 is also configured to pierce through the pierceable seal of the entity, when the entity is being coupled to the connector device 100. For example, the second end projection 162 of the at least one second fluid conduit 160 is configured to pierce through the elastomeric stopper 210 of the test tube 200, when the test tube 200 is being coupled to the connector device 100. In an embodiment, the second end projection 162 comprises a tapered or sharpened tip 164 for piercing the elastomeric stopper 210 of the test tube 200. In a further embodiment, the second end projection 162 comprises a bevelled end, for example, shaped like a needle, for piercing the elastomeric stopper 210 of the test tube 200.

In this embodiment, the first fluid conduit 150 and the second fluid conduit 160 are arranged parallel to each other. Further, one or both of the at least one first fluid conduit 150 or the at least one second fluid conduit 160 may be constituted by a metal tube.

The test tube port 120 and the syringe port 120 of the adapter body 110 are, respectively, adapted for coupling with the test tube 200 and the syringe 400. When the test tube 200 is coupled to the test tube port 120, as seen in FIGS. 5 to 7, the first end projection 152 of the at least one first fluid conduit 150 and the second end projection 162 of the at least one second fluid conduit 160 pierce the elastomeric stopper 210 of the test tube 200 such that the first end projection 152 and the second end projection 162 are received in the test tube 200. The at least one second fluid conduit 160 enables the fluid communication between the test tube 200 and the gas inlet port 140 through channel 144.

Further, as also seen in FIG. 5, when the syringe 400 is coupled to the syringe port 130 of the connector device 100 (to which the test tube 200 is coupled), the at least one first fluid conduit 150 enables the fluid communication between the test tube 200 and the syringe 400. When the plunger 410 of the syringe 400 is actuated to draw the blood mass 202 contained in the test tube 200, the at least one first fluid conduit 150 facilitates drawing the blood mass 202 from the test tube 200 into the syringe 400 so as to mix the blood mass 202 with the coagulating agent 420 contained in the syringe 400, thereby forming the coagulating blood mass 206, as seen in FIG. 6. Simultaneously, the at least one second fluid conduit 160 facilitates the fluid communication between the atmosphere/external source of gas and the test tube 200 so that the gas is introduced into the test tube 200 when the blood mass 202 is drawn from the test tube 200.

Without derogating from the general scope of the teaching of present disclosure, the connector device 100, along with the components thereof, for example, the adapter body 110, the gas inlet port 140, the at least one first fluid conduit 150 and the at least one second fluid conduit 160, facilitates easy and convenient withdrawal of the blood mass 202 from the test tube 200, against the vacuum 204 that is present and/or develops within the test tube 200. Particularly, the at least one second fluid conduit 160 permits withdrawal of the blood mass 202 from the test tube 200 while introducing gas, for example, atmospheric air, thereinto.

As illustrated in the exemplary embodiment of FIGS. 1, 2, 5 and 6, the test tube port 120 and the syringe port 130 of the adapter body 110 of the connector device 100 are disposed opposite to each other. Further, in an embodiment, the test tube adapter 120 is aligned co-axially with the syringe adapter 130. However, without derogating from the general scope of teachings of the present disclosure, a person skilled in the art can modify the constructional features of the connector device 100 such that the test tube port 120 and the syringe port 130 can be provided at any angle with respect to each other, not necessarily 180 degrees apart (i.e., opposite to each other). In said modifications, the test tube 200 and the syringe 400 can be coupled with the connector device 100 at any desired angle, not necessarily 180 degrees apart (i.e., opposite to each other).

Further, in the embodiment where the test tube adapter 122 is formed in shape of the hollow cylinder 124 having the open end 126, the first end projection 152 and the second end projection 162 of the at least one first fluid conduit 150 and the at least one second fluid conduit 160, respectively, project into an interior of the hollow cylindrical test tube adapter 122. Further, when the test tube 200 is coupled with the connector device 100, the test tube adapter 122 receives the stopper-sealed end of the test tube 200 in a manner such that the first and second end projections 152, 162 pierce the elastomeric stopper 210 of the test tube 200. Also, the hollow cylinder of the test tube adapter 122 provides support to the test tube 200 and holds the test tube 200 in a desired position.

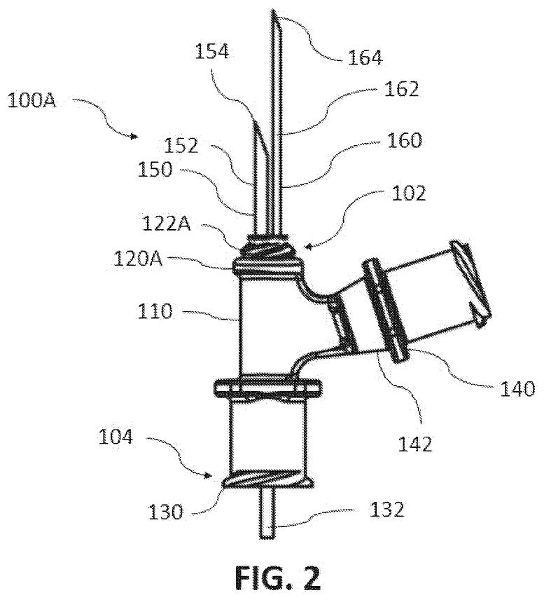

While the coupling of the syringe 400 and the test tube 200 to the connector device 100 and the drawing of blood into the syringe is described in FIGS. 5-6 in relation to the embodiment thereof shown in FIG. 1, use may also be made with a connector device 100A of the embodiment shown in FIGS. 2 and 3, which differs from device 100 in that test tube port 120A of connector device 100A does not have a cylindrical tube receptacle such as cylinder 124, but rather a screw-threaded adapter 122A that is configured for coupling with a matching closure with an internal (female-type) screw threading that can be screw-fitted thereto. Other elements of connector device 100A are substantially the same as those of connector device 100 and are indicated by the same reference numerals.

In other embodiment, the coupling between the test tube 200 and the test tube port 120 may be achieved by a variety of different means and, even just through permitting only the first and second end projections 150,160 to pierce the stopper with the coupling being achieved in this manner without any other coupling element.

Further, in accordance with the present disclosure, a system 501 for coagulating a blood mass, for example, the blood mass 202, is disclosed. An exemplary system 501 for coagulating the blood mass 200 is shown in FIGS. 5 and 6. In an embodiment, the system 501 comprises a test tube, for example, the test tube 200, a syringe 600 and a connector device 500 couplable with the test tube 200 and the syringe 600. The test tube 200 may comprise any known test tube known in the medical field. Typically, the test tube 200 is formed of a medical grade material (for example, glass, plastic, another polymeric mater, etc.) and in shape of a cylindrical tube having an open end. The open end of the test tube is sealed by a closure 208 that includes an elastomeric stopper 210 that may be made of any suitable medical grade elastomeric material such as rubber. Further, the test tube 200 is adapted to contain the blood mass 202 and hold a vacuum 204 (as known, the vacuum being the driving force for drawing blood therein, e.g., for venous blood withdrawal from a subject). The test tube 200 may be such used for blood collection. The blood mass 202 is whole blood, typically, an autologous blood, withdrawn from a subject, for example, a human or non-human animal. The blood mass 202 is collected in the test tube 200 that typically holds the vacuum 204, maintained by the elastomeric stopper 210 of the test tube 200.

The system 501 comprises the connector device 500, that in an exemplary embodiment, have all the structural and functional features similar to that of the connector device 100, 100A described above, and elements in FIGS. 5-6 with a similar function to those of FIGS. 1-3, are indicated by the same reference numeral shifted by 400. For example, the elements 510, 550 and 560 in FIGS. 5 and 6 have the same functionality as elements 110, 150 and 160 in FIGS. 1-3, respectively. Similarly, elements of the syringe 600 with a similar function to those of FIG. 4, are indicated by the same reference numeral shifted by 200. For example, the elements 602 and 620 have the same functionality as elements 402 and 420 in FIG. 4, respectively. The reader can thus refer to the description above for explanation of structure or function of such elements.

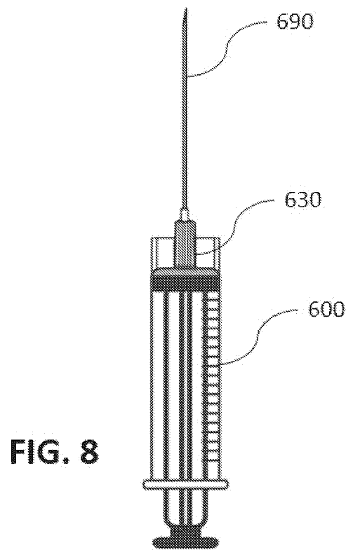

In an embodiment, the system 501 comprises an elongated application tube or a needle 690, as shown in FIG. 8, adapted to be coupled or fixed to an outlet port 630 of the syringe 600. Said elongated application tube or the needle 690 may be any suitable tube or needle used in medical procedures for applying or injecting or transferring or administering the contents, for example, the coagulating blood mass 206, contained within the syringe 600 to a site of need, for example, a wound, in a body of the subject. In a typical embodiment, the coagulating blood mass 206 is applied or transferred to the wound while the coagulating blood mass 206 is still flowable, and hence injectable out of the syringe 600. The coagulating blood mass 206, depending on the nature and concentration of the coagulating agent 620, may become too viscous for injection after a time period of about 4 to 15 minutes, and accordingly, the coagulating blood mass 206 is applied or transferred out of the syringe 600 within a time period of about 1 to 10 minutes, or up to about 3 or 4 minutes, from mixing of the blood mass 202 with the coagulating agent 620. In an embodiment, the system 501 further comprises one or more blood mass collection elements (not shown), for example, needles, etc., for withdrawing the blood mass 202 from the subject and transferring the blood mass 202 to/into the test tube 200.

Further, the present disclosure provides a method of coagulating the blood mass 202. Without derogating from the general scope of teachings of the present disclosure, the method of coagulating the blood mass 202 may be performed by the system 501 of coagulating the blood mass 202 described above. The method comprises coupling the syringe 600 containing the coagulating agent 620, to the syringe port 530 of the connector device 500. The method further comprises coupling the stopper-sealed test tube 200 containing a volume of the blood mass 202, to the test tube port 520 of the connector device 500, such that the first and second end projections 552, 562 of the at least one first fluid conduit 550 and the at least one second fluid conduit 560, respectively, pierce the elastomeric stopper 210 of the test tube 200. The method furthermore comprises drawing the blood mass 202 from the test tube 200 into the syringe 600, through the at least one first fluid conduit 550, for mixing the blood mass 202 with the coagulating agent 620 to form the coagulating blood mass 206. In an embodiment, the method comprises, prior to coupling the test tube 200 with the connector device 500, withdrawing the blood mass 202, by one or more blood mass collection elements known per se, from the subject and transferring the blood mass 202 to/into the test tube 200.

The coagulating blood mass 206 so formed in the syringe 600, by the method of coagulating the blood mass 202, may be applied or injected onto a wound of the subject for treating the wound by forming a blood coagulant on the wound. The coagulating blood mass 206 is applied or injected through the elongated application tube or the needle 690 couplable with the syringe port 630.

Further, in accordance with the present disclosure, a wound treatment system is disclosed. By an embodiment of the present disclosure, the wound treatment system comprises the elements of the system 501 of FIGS. 5 to 6.

Further, the present disclosure provides a method of treating a wound. Without derogating from the general scope of teaching of the present disclosure, the method of treating the wound may be performed by the wound treatment system described above.

The elements of the system 501 of FIGS. 5-6 and 8 may also be part of a kit of parts also provided by this disclosure. The kit of parts may also comprise blood withdrawal elements for withdrawing a blood mass from the subject as well as others.

The various embodiments of the present disclosure have been described above with reference to the accompanying schematic drawings. The present disclosure is not limited to the illustrated embodiments; rather, these embodiments are intended to fully and completely disclose the subject matter of the disclosure to those skilled in this art. In the drawings, like numbers refer to like elements throughout. Thicknesses and dimensions of some components may be exaggerated for clarity.

Herein, the terms “attached”, “connected”, “interconnected”, “contacting”, “mounted”, “coupled” and the like can mean either direct or indirect attachment or contact between elements, unless stated otherwise.

Well-known functions or constructions may not be described in detail for brevity and/or clarity. As used herein the expression “and/or” includes any and all combinations of one or more of the associated listed items.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises”, “comprising”, “includes” and/or “including” when used in this specification, specify the presence of stated features, operations, elements, and/or components, but do not preclude the presence or addition of one or more other features, operations, elements, components, and/or groups thereof.

While considerable emphasis has been placed herein on the particular features of this disclosure, it will be appreciated that various modifications can be made, and that many changes can be made in the described embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

REFERENCE NUMERALS

| | REFERRAL |
|---|---|
| PARTICULARS | NUMERAL |
| Connector Device | 100, 100A |
| First End | 102 |
| Second End | 104 |
| Adapter Body | 110 |
| Test Tube Port | 120, 120A |
| Test Tube Adapter | 122, 122A |
| Cylinder | 124 |
| Open End | 126 |
| Syringe Port | 130 |
| Syringe Adapter | 132 |
| Gas Inlet Port | 140 |
| Body | 142 |
| Channel | 144 |
| Filter | 146 |
| First Fluid Conduit | 150 |
| First End Projection | 152 |
| Tapered or Sharpened Tip | 154 |
| Second Fluid Conduit | 160 |
| Second End Projection | 162 |
| Tapered or Sharpened Tip | 164 |
| Test tube | 200 |
| Blood mass | 202 |
| Vacuum | 204 |
| Coagulating Blood Mass | 206 |
| Closure | 208 |
| Elastomeric Stopper | 210 |
| Syringe | 400 |
| Syringe Barrel | 402 |
| Front End | 404 |
| Rear End | 406 |
| Walls | 408 |
| Plunger | 410 |
| Piston | 412 |
| Coagulating Agent | 420 |
| Connector Device | 500 |
| System | 501 |
| Adapter Body | 510 |
| Test Tube Port | 520 |
| Test Tube Adapter | 522 |
| Hollow Cylinder | 524 |
| Syringe Port | 530 |
| Syringe Adapter | 532 |
| Gas Inlet Port | 540 |
| First Fluid Conduit | 550 |
| First End Projection | 552 |
| Tapered or Sharpened Tip | 554 |
| Second Fluid Conduit | 560 |
| Second End Projection | 562 |
| Tapered or Sharpened Tip | 564 |
| Syringe | 600 |
| Syringe Barrel | 602 |
| Plunger | 610 |
| Piston | 612 |
| Coagulating Agent | 620 |
| Outlet Port | 630 |
| Elongated Application Tube/Needle | 690 |

### EQUIVALENTS

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the general scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the general scope of the disclosure, unless there is a statement in the specification specific to the contrary.

1. A system for preparing a coagulating blood volume, comprising:
a syringe accommodating a blood coagulating agent; and
a connector device that comprises
an adapter body having (i) a test tube port for coupling with a test tube containing a whole blood mass, (ii) a syringe port for coupling with the syringe, and (iii) a gas inlet port;
at least one first fluid conduit extending through the adapter body between the test tube port and the syringe port, the at least one first fluid conduit comprising a first end projection at the test tube port configured for piercing through a stopper of the test tube; and
at least one second fluid conduit extending through the adapter body between the test tube port and the gas inlet port, the at least one second fluid conduit comprising a second end projection at the test tube port configured for piercing through the stopper of the test tube;
when a test tube is coupled to the test tube port and a syringe is coupled to the syringe port, the at least one first fluid conduit enables fluid communication between the test tube and the syringe, and the at least one second fluid conduit enables fluid communication between the test tube and the gas inlet port.

2. The system as claimed in claim 1, comprising a test tube for containing a volume of whole blood.

3. The system as claimed in claim 1, wherein the coagulating agent contained in the syringe is a dry particulate matter.

4. (canceled)

5. The system as claimed in claim 1, wherein the coagulating agent is one or more first agents that induce blood coagulation and/or one or more second anti-anticoagulants that counteracts action of anti-coagulants.

6. The system as claimed in claim 1, wherein the test tube holds a vacuum.

7. A method for preparing a coagulating a blood mass, comprising:
coupling a syringe containing a coagulating agent to a syringe port of a connector device that comprises
an adapter body having (i) a test tube port for coupling with a test tube containing a whole blood mass, (ii) a syringe port for coupling with the syringe, and (iii) a gas inlet port,
at least one first fluid conduit extending through the adapter body between the test tube port and the syringe port, the at least one first fluid conduit comprising a first end projection at the test tube port configured for piercing through a stopper of the test tube, and comprises
at least one second fluid conduit extending through the adapter body between the test tube port and the gas inlet port, the at least one second fluid conduit comprising a second end projection at the test tube port configured for piercing through the stopper of the test tube; coupling a test tube containing a volume of blood to the test tube port of the connector device, such that the first and second end projections of the at least one first fluid conduit and the at least one second fluid conduit, respectively, pierce a stopper of the test tube; and comprising
drawing the blood mass from the test tube into the syringe, through the at least one first fluid conduit, for mixing the blood mass with the coagulating agent to form a coagulating blood mass.

8. The method as claimed in claim 7, comprising, prior to coupling the test tube with the connector device,
withdrawing blood from a subject and transferring the blood to the test tube.

9. (canceled)

10. A wound treatment system, comprising:
a syringe accommodating a coagulating agent;
a connector device that comprises
an adapter body having (i) a test tube port for coupling with a test tube containing a whole blood mass, (ii) a syringe port for coupling with the syringe, and (iii) a gas inlet port,
at least one first fluid conduit extending through the adapter body between the test tube port and the syringe port, the at least one first fluid conduit comprising a first end projection at the test tube port configured for piercing through a stopper of the test tube, and comprises
at least one second fluid conduit extending through the adapter body between the test tube port and the gas inlet port, the at least one second fluid conduit comprising a second end projection at the test tube port configured for piercing through the stopper of the test tube; and comprising
an elongated application tube or a needle configured for coupling with the syringe and for transferring the coagulating blood mass to a site of need in a body of a subject.

11. (canceled)

12. The system as claimed in claim 10, comprising:
one or more blood mass collection elements for withdrawing the blood mass from the subject and transferring the blood mass to the test tube.

13. The system as claimed in claim 10, wherein the coagulating agent contained in the syringe is a dry particulate matter.

14. (canceled)

15. The system as claimed in claim 10, wherein the coagulating agent is one or more first agents that induce blood coagulation and/or one or more second anti-anticoagulants that counteracts action of anti-coagulants.

16. (canceled)

17. A method of treating a wound, comprising:
coupling a syringe containing a coagulating agent to the syringe port of the connector device that comprises
an adapter body having (i) a test tube port for coupling with a test tube containing a whole blood mass, (ii) a syringe port for coupling with the syringe, and (iii) a gas inlet port,
at least one first fluid conduit extending through the adapter body between the test tube port and the syringe port, the at least one first fluid conduit comprising a first end projection at the test tube port configured for piercing through a stopper of the test tube, and comprises
at least one second fluid conduit extending through the adapter body between the test tube port and the gas inlet port, the at least one second fluid conduit comprising a second end projection at the test tube port configured for piercing through the stopper of the test tube;
coupling a test tube containing a volume of blood to the test tube port of the connector device, such that the first and second end projections of the at least one first fluid conduit and the at least one second fluid conduit, respectively, pierce a stopper of the test tube;
drawing the blood mass from the test tube into the syringe, through the at least one first fluid conduit, for mixing the blood mass with the coagulating agent to form a coagulating blood mass; and comprising
transferring the coagulating blood mass to a site of need in a body of a subject.

18. The method as claimed in claim 17, wherein transferring the coagulating blood mass in the body of the subject comprises transferring the coagulating blood mass through an elongated application tube or a needle configured for coupling with the syringe.

19. The method as claimed in claim 17, comprising, prior to coupling the test tube with the connector device,
withdrawing the blood mass from the subject and transferring the blood mass to the test tube.

20-29. (canceled)
